(19) United States
(12) Patent Application Publication
(10) Pub. No.: US 2023/0295496 A1
(43) Pub. Date: Sep. 21, 2023

(54) PROCESS FOR THE PREPARATION OF FLUXED UP-CONVERSION PHOSPHORS

(71) Applicant: Evonik Operations GmbH, Essen (DE)

(72) Inventors: Simone Schulte, Essen (DE); Michael Huth, Maintal (DE); Stefan Fischer, Soest (DE); Christina Janke, Essen (DE); Juri Tschernjaew, Aschaffenburg (DE)

(73) Assignee: Evonik Operations GmbH, Essen (DE)

(21) Appl. No.: 18/185,367

(22) Filed: Mar. 16, 2023

(30) Foreign Application Priority Data
Mar. 17, 2022 (EP) 22162615.3

(51) Int. Cl. C09K 11/77; C09D 5/14; C09D 7/61

(52) U.S. Cl. CPC C09K 11/77062; C09D 5/14; C09D 7/61; C08K 3/34

(57) ABSTRACT

A process can be used for the preparation of an up-conversion phosphor of the general formula (I)

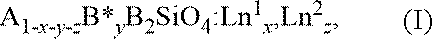

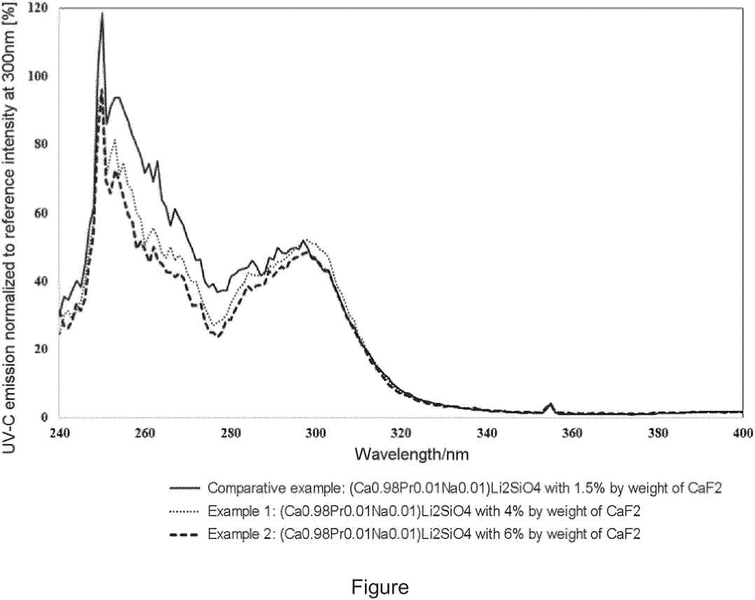

The process involves providing i) at least one lanthanoid salt, ii) a silicate or a silicon dioxide, iii) at least one alkaline earth metal salt and at least one alkali metal salt, and iv) at least one flux. The process then involves either mixing components i), ii), iii) and iv) by grinding to obtain a mixture; or mixing components i), ii), iii) and iv) in an organic polar or nonpolar solvent that is not a protic solvent by grinding to obtain a mixture, and precalcining the mixture. The process further involves calcining the mixture, and obtaining a silicate-based up-conversion phosphor of the general formula (I), preferably after cooling the material. At least 3.5% by weight of flux is used, based on the total amount of the reactants.

### CROSS-REFERENCE TO RELATED APPLICATIONS

This application claims priority to European Application No. 22162615.3, filed on Mar. 17, 2022, the content of which is hereby incorporated by reference in its entirety.

### BACKGROUND OF THE INVENTION

### Field of the Invention

The invention relates to a process for the preparation of fluxed up-conversion phosphors, to the fluxed up-conversion phosphor and to the use thereof in coatings having an antimicrobial property.

### Description of Related Art

Every day, humans are exposed to millions of microorganisms such as bacteria, fungi and viruses. Many of these microorganisms are useful or even necessary. Nevertheless, as well as these less harmful representatives, there are also disease-causing or even deadly bacteria, fungi and viruses.

Microorganisms can be transmitted through daily interaction with other people and contact with articles that have been used by others. Surfaces are given an antimicrobial finish especially in hygiene-sensitive areas. Fields of use are in particular surfaces of medical devices and consumer articles in hospitals, and in outpatient health and welfare facilities. In addition to these, there are surfaces in the public sphere, in the food and drink sector and in animal keeping. The spread of pathogenic microorganisms is a great problem nowadays in the care sector and in medicine, and wherever humans associate in an enclosed space. A particular risk at present is the increased occurrence of what are called multiresistant germs that have become insensitive to standard antibiotics.

In order to reduce the risk of spread of pathogens via contact surfaces, in addition to standard hygiene measures, antimicrobial technologies and materials are being utilized. Chemical substances or the use of physical methods can have a critical influence on the process of propagation of microorganisms. The physical methods include, for example, heat, cold, radiation or ultrasound, etc. Among the chemical methods, halogens, metal ions, organic compounds and dyes, toxic gases, etc., are known.

Even though chemical and physical methods are extremely effective in the destruction of microorganisms in most cases, they have only a short-lived effect, chemical methods promote the development of resistances and are unsuitable for some applications under some circumstances since they lead to destruction of the surfaces to be protected. The greatest disadvantage, however, specifically in the case of chemical organic substances, is the hazard or toxicity to man. Particular substances, for example formaldehyde, which found use as disinfectant for many years, are now suspected of causing cancer or of being extremely harmful to the environment.

Surfaces with antimicrobial action can make a crucial contribution to the solution of these problems. The standard processes nowadays for generation of such antimicrobial properties make use predominantly of active ingredients incorporated into the material, for example silver particles, copper particles, metal oxides thereof or quaternary ammonium compounds. This frequently involves processing the antimicrobial metals, metal oxides or metal oxide mixtures to give nanoparticles and then mixing them into paints, coatings or polymer materials. The broad use of metal particles is questionable since it is barely possible to assess the long-term effect of this heavy metal on man and the environment.

For example, WO 2019/197076 discloses particles finished with a layer containing both antimony tin oxide and manganese oxide. The person skilled in the art is aware that the antimicrobial surfaces are produced on account of the electrochemical characteristics of metals which, in the presence of moisture, develop microscale galvanic cells and, by virtue of the microscale electrical fields, germ-killing action.

It is likewise known that UV radiation can be used in medicine or in hygiene, in order, for example, to disinfect water, gases or surfaces. For instance, UV radiation has long been used in drinking water treatment to reduce the number of facultatively pathogenic microorganisms in the water. This is preferably done using UV-C radiation in the wavelength range between 200 nm and 280 nm. The use of electromagnetic radiation with different wavelengths should take account of the different absorption of the different proteins, the amino acids/nucleic acids (e.g. DNA or RNA) present in microorganisms, tissues or cells, and peptide bonds between the individual acids. For instance, DNA/RNA has good absorption of electromagnetic radiation in the wavelength range between 200 nm and 300 nm, and particularly good absorption between 250 nm and 280 nm, and so this radiation is particularly suitable for inactivation of DNA/RNA. It is thus possible to inactivate pathogenic microorganisms (viruses, bacteria, yeasts, moulds inter alia) with such irradiation. Depending on the duration and intensity of the irradiation, the structure of DNA or RNA can be destroyed. Thus, metabolically active cells are inactivated and/or their capacity for propagation can be eliminated. What is advantageous about irradiation with UV radiation is that the microorganisms are unable to develop resistance thereto. However, these physical methods require specific apparatuses and generally have to be repeated regularly by trained personnel, which makes it difficult for these methods to be used widely.

Furthermore, as well as direct irradiation with electromagnetic radiation from the wavelength range of UV radiation, the exploitation of the “up-conversion” effect is also known. This uses phosphor particles with which electromagnetic radiation having wavelengths above UV radiation, especially visible light or infrared radiation, can be converted to electromagnetic radiation having shorter wavelength, such that it is possible to achieve the emission of radiation having the desired wavelength by the individual phosphor particles.

DE 102015 102 427 relates to a body that emits electromagnetic radiation in the wavelength range of UV light. Phosphor particles are embedded in the body in a near-surface region within the material from which the body is formed or in a coating on the body. All that is stated here in general terms is that the phosphor particles are added directly to a coating to be formed on the material in the course of processing, where the particular material should have a suitable consistency or viscosity. DE 10 2015 102 427 is silent with regard to suitable polymers and additives.

US 2009/0130169 A1 and WO 2009/064845 A2 describe phosphors that can be introduced into polyvinyl chlorides, acryloylbutadienes, polyolefins, polycarbonates, polystyrenes or nylon, which kill pathogenic microorganisms by virtue of the up-conversion property of the phosphors. These are phosphors that are prepared at a temperature of 1800-2900° C. While US 2009/0130169 A1 and WO 2009/064845 A2 do disclose a composition comprising said phosphors having an asserted antimicrobial action, they do not demonstrate either evidence of the up-conversion property or microbiological experiments. The process disclosed in these documents does not result in a phosphor having an up-conversion property, but instead in an amorphous and glass-like product.

Moreover, US 2009/0130169 A1 and WO 2009/064845 A2 are silent as regards the compatibility of the component in the coating composition and the properties of the coating surfaces, such as the paint surfaces, for example. However, the appearance of coating surfaces is paramount for the consumer.

The demands on coatings and paints are diverse. In principle, coating layers or paint coatings have two tasks or functions: the protective and the decorative function. If merely the term “coating layer” should be stated below, both types of coating are intended. They decorate, protect and preserve materials such as wood, metal or plastic. Accordingly, bright and glossy coat layers are required on the one hand, and a continuous coat layer on the other hand for assurance of chemical and mechanical stability, a certain glide over the coatings or a particular feel.

In contrast to WO 2009/064845 A2, the patent application PCT/EP2020/077798 discloses phosphors exhibiting up-conversion and the preparation thereof. On irradiation with electromagnetic radiation having lower energy and longer wavelength in the range from 2000 nm to 400 nm, in particular in the range from 800 nm to 400 nm, such phosphors may emit electromagnetic radiation having higher energy and shorter wavelength in the range from 400 nm to 100 nm, preferably in the range from 300 nm to 200 nm, with the result that they are suitable for use as antimicrobial phosphors in coating layers.

For instance, EP 3929254 describes a composition comprising at least one film-forming polymer, at least one up-conversion phosphor according to the teaching of PCT/EP2020/077798, optionally at least one additive and optionally at least one curing agent. It was shown that coating layers comprising these phosphors have antimicrobial action without the other properties, in particular the storage stability, being significantly impaired.

However, it was also found that the phosphors prepared by a process according to PCT/EP2020/077798 exhibit an inhomogeneous particle size distribution, which presents a particular challenge when incorporating these phosphors into a coating matrix. Even though the teaching of EP 3929254 leads to antimicrobial coating layers, it would additionally be desirable to be able to increase the intensity of the emission of the phosphors.

The as-yet unpublished European patent application EP 21167984.0 proposed using a phosphor which has been prepared using at least one halogen-containing flux for the production of coatings having an antimicrobial property, comprising
at least one film-forming polymer,
optionally at least one additive,
optionally at least one curing agent,
at least one up-conversion phosphor of the general formula (I)

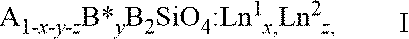

with
   x = 0.0001 - 0.0500;
   z = 0.0000 or z = 0.0001 to 0.3000 with the proviso that: y = x + z;
   A being selected from the group consisting of Mg, Ca, Sr and Ba;
   B being selected from the group consisting of Li, Na, K, Rb and Cs;
   B* being selected from the group consisting of Li, Na and K, where B is the same as B* or B is not the same as B*, and preferably B and B* are not the same;
   Ln¹ being selected from the group consisting of praseodymium (Pr), erbium (Er) and neodymium (Nd);
   Ln² being selected from gadolinium (Gd).

In this case, at most 3.5% by weight of flux is used, based on the total amount of the reactants.

### SUMMARY OF THE INVENTION

Proceeding from the as-yet unpublished European patent application EP21167984.0 it would therefore be desirable to improve the phosphor and additionally be able to optimize the process for the preparation thereof.

The person skilled in the art is aware of a great number of fluxes of all kinds from the prior art, such as halides, carbonates, sulfates, oxides and borates of, where respectively applicable, ammonium, lithium, sodium, potassium, rubidium, caesium, magnesium, calcium, strontium, barium, lead, lanthanum, lutetium, aluminium, bismuth and boric acid. Also known are their applications in the field of metallurgy, for example for accelerating crystal growth or suppressing the formation of extraneous phases.

Treatment with fluxes is also called fluxing, that is to say the product has been fluxed.

Surprisingly, the object was achieved by a process as described below.

A process is proposed for the preparation of an up-conversion phosphor of the general formula (I)

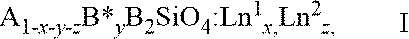

with
x = 0.0001 - 0.0500;
z = 0.0000 or z = 0.0001 to 0.3000 with the proviso that: y = x + z;
A being selected from the group consisting of Mg, Ca, Sr and Ba;
B being selected from the group consisting of Li, Na, K, Rb and Cs;
B* being selected from the group consisting of Li, Na and K, where B is the same as B* or B is not the same as B*, and preferably B and B* are not the same;
Ln¹ being selected from the group consisting of praseodymium (Pr), erbium (Er) and neodymium (Nd);
Ln² being selected from gadolinium (Gd),
comprising the following steps:
i) providing at least one lanthanoid salt selected from lanthanoid nitrates, lanthanoid carbonates, lanthanoid carboxylates, preferably lanthanoid acetates, lanthanoid sulfates, lanthanoid oxides, particularly preferably Pr₆O₁₁ and/or Gd₂O₃, where the lanthanoid ions in the lanthanoid oxides or lanthanoid salts are selected from praseodymium, gadolinium, erbium, neodymium and, for co-doping, at least two of these,
ii) providing a silicate, preferably a silicate salt, particularly preferably an alkali metal salt of the silicate, or a silicon dioxide,
iii) providing at least one alkaline earth metal salt and at least one alkali metal salt, preferably an alkali metal silicate or an alkali metal carbonate, selected from a lithium salt or a lithium compound and optionally selected from a sodium salt and potassium salt, preferably the salt of the lithium salt, preferably a lithium silicate, particularly preferably a lithium carbonate, a calcium carbonate and a sodium carbonate,
iv) providing at least one flux from the group of the ammonium halides, preferably ammonium chloride, alkali metal halides, preferably sodium chloride, sodium fluoride, sodium bromide, lithium fluoride or lithium chloride, alkaline earth metal halides, preferably calcium chloride or calcium fluoride, and lanthanoid halides, preferably praseodymium fluoride or praseodymium chloride,
a) mixing components i), ii), iii) and iv) by means of grinding to obtain a mixture, or
b) mixing components i), ii) and iii) and iv) in an organic polar or nonpolar solvent that is not a protic solvent by means of grinding to obtain a mixture;
c) precalcining the mixture from b) at 600 to 1000° C., also to remove the organic component, for at least 1 h, preferably not less than 2 h, under air atmosphere to obtain a precalcined mixture, optionally cooling to room temperature,
d) calcining the mixture from a) or the precalcined mixture from c) at a temperature from 600 to < 1000° C., preferably at 650 to 900° C., for at least 3 h, preferably for at least 12 h,
e) obtaining a silicate-based up-conversion phosphor of the general formula (I), preferably after cooling the material,
wherein at least 3.5% by weight of flux is used, based on the total amount of the reactants.

The invention also includes the following embodiments:

1. Process for the preparation of an up-conversion phosphor of the general formula (I)

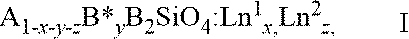

with
x = 0.0001 - 0.0500;
z = 0.0000 or z = 0.0001 to 0.3000 with the proviso that: y = x + z;
A being selected from the group consisting of Mg, Ca, Sr and Ba;
B being selected from the group consisting of Li, Na, K, Rb and Cs;
B* being selected from the group consisting of Li, Na and K, where B is the same as B* or B is not the same as B*, and preferably B and B* are not the same;
Ln¹ being selected from the group consisting of praseodymium (Pr), erbium (Er) and neodymium (Nd);
Ln² being selected from gadolinium (Gd),
comprising the following steps:
i) providing at least one lanthanoid salt selected from lanthanoid nitrates, lanthanoid carbonates, lanthanoid carboxylates, preferably lanthanoid acetates, lanthanoid sulfates, lanthanoid oxides, particularly preferably Pr₆O₁₁ and/or Gd₂O₃, where the lanthanoid ions in the lanthanoid oxides or lanthanoid salts are selected from praseodymium, gadolinium, erbium, neodymium and, for co-doping, at least two of these,
ii) providing a silicate, preferably a silicate salt, particularly preferably an alkali metal salt of the silicate, or a silicon dioxide,
iii) providing at least one alkaline earth metal salt and at least one alkali metal salt, preferably an alkali metal silicate or an alkali metal carbonate, selected from a lithium salt or a lithium compound and optionally selected from a sodium salt and potassium salt, preferably the salt of the lithium salt, preferably a lithium silicate, particularly preferably a lithium carbonate, a calcium carbonate and a sodium carbonate,
iv) providing at least one flux from the group of the ammonium halides, preferably ammonium chloride, alkali metal halides, preferably sodium chloride, sodium fluoride, sodium bromide, lithium fluoride or lithium chloride, alkaline earth metal halides, preferably calcium chloride or calcium fluoride, and lanthanoid halides, preferably praseodymium fluoride or praseodymium chloride,
a) mixing components i), ii), iii) and iv) by means of grinding to obtain a mixture, or
b) mixing components i), ii) and iii) and iv) in an organic polar or nonpolar solvent that is not a protic solvent by means of grinding to obtain a mixture;
c) precalcining the mixture from b) at 600 to 1000° C., also to remove the organic component, for at least 1 h, preferably not less than 2 h, under air atmosphere to obtain a precalcined mixture, optionally cooling to room temperature,
d) calcining the mixture from a) or the precalcined mixture from c) at a temperature from 600 to < 1000° C., preferably at 650 to 900° C., for at least 3 h, preferably for at least 12 h,
e) obtaining a silicate-based up-conversion phosphor of the general formula (I), preferably after cooling the material,
characterized in that at least 3.5% by weight of flux is used, based on the total amount of the reactants.

2. Process according to embodiment 1, characterized in that the amount of flux is not more than 50.0% by weight, preferably not more than 10.0% by weight, particularly preferably not more than 4.0% by weight, based on the total amount of the reactants.

3. Process according to either of the preceding embodiments, characterized in that the calcination (step d) is conducted under air atmosphere.

4. Process according to any of the preceding embodiments, characterized in that the lanthanoid is praseodymium.

5. Process according to any of the preceding embodiments, characterized in that the alkali metals are sodium or lithium.

6. Process according to any of the preceding embodiments, characterized in that the alkaline earth metal is calcium.

7. Process according to any of the preceding embodiments, characterized in that the phosphor has been doped with praseodymium.

8. Up-conversion phosphor of the general formula (I)

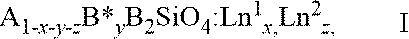

with
x = 0.0001 - 0.0500;
z = 0.0000 or z = 0.0001 to 0.3000 with the proviso that: y = x + z;
A being selected from the group consisting of Mg, Ca, Sr and Ba;
B being selected from the group consisting of Li, Na, K, Rb and Cs;
B* being selected from the group consisting of Li, Na and K, where B is the same as B* or B is not the same as B*, and preferably B and B* are not the same;
Ln¹ being selected from the group consisting of praseodymium (Pr), erbium (Er) and neodymium (Nd);
Ln² being selected from gadolinium (Gd), obtainable by a process according to any of the preceding embodiments, characterized in that it has a specific surface area determined by gas absorption according to Brunauer, Emmett and Teller (BET) of 1 to 500 m²/g, preferably 5 - 250 m²/g, particularly preferably 10 - 100 m²/g, measured to ISO 9277, DIN 66131.

9. Phosphor according to embodiment 8, characterized in that the phosphor has been doped with praseodymium and co-doped with gadolinium.

10. Phosphor according to any of embodiments 8 - 9, characterized in that the phosphor is a solidified melt composed of crystalline silicates or of crystalline silicates doped with lanthanoid ions, comprising at least one alkali metal ion and at least one alkaline earth metal ion, preferably in that the crystalline silicates have been doped with praseodymium and optionally co-doped with gadolinium.

11. Phosphor according to any of embodiments 8 - 10, characterized in that the phosphor is at least partially crystalline.

12. Phosphor according to any of embodiments 8 - 11, characterized in that the phosphor is selected from compounds of the general formula (Ia)

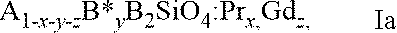

with A being selected from the group consisting of Mg, Ca, Sr, Ba;
B being selected from the group consisting of Li, Na, K, Rb and Cs;
B* being selected from the group consisting of Li, Na and K, where B is the same as B* or B is not the same as B*, and preferably B and B* are not the same;
x = 0.0001 - 0.0500;
z = 0.0000 or z = 0.0001 to 0.3000 with the proviso that: y = x + z.

13. Phosphor according to any of embodiments 8 - 11, characterized in that the phosphor is selected from compounds of the general formula (II)

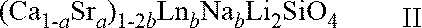

where:
Ln is selected from the group consisting of praseodymium, gadolinium, erbium, neodymium, preferably praseodymium;
a = 0.0000 to 1.0000, preferably 0.0000 to 0.1000, especially 0.0000;
b = 0.0001 to 0.5000, preferably 0.0001 to 0.1000, especially 0.0050 to 0.0500.

14. Phosphor according to any of embodiments 8 - 11, characterized in that the phosphor is selected from compounds of the general formula (IIa)

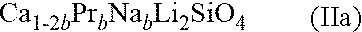

with b = 0.0001 to 1, preferably 0.0001 to 0.1, especially 0.005 to 0.0500.

15. Phosphor according to any of embodiments 8 - 14, characterized in that the phosphor is Ca_{0.98}Pr_{0.01}Na_{0.01}Li₂SiO₄ or Ca_{0.94}Pr_{0.03}Na_{0.03}Li₂SiO₄ or Ca_{0.90}Pr_{0.05}Na_{0.05}Li₂SiO₄.

16. Phosphor according to any of embodiments 8 - 16, characterized in that the phosphor according to formula (II) has XRPD signals in the range from 23° 2θ to 27° 2θ and from 34° 2θ to 39.5° 2θ.

17. Use of the phosphors according to any of embodiments 8 - 17 for the production of coatings having an antimicrobial property comprising
at least one film-forming polymer,
optionally at least one additive,
optionally at least one curing agent.

### BRIEF DESCRIPTION OF THE DRAWING

The FIGURE shows an emission spectrum for Examples 1 and 2 and for the comparative example.

### DETAILED DESCRIPTION OF THE INVENTION

It was found that the up-conversion phosphor has improved properties, such as for example the specific surface area.

A further advantage of the invention is the aspect of operational safety. Increasing the amount of flux resulted, completely unexpectedly, in it being possible to dispense with the use of reducing gases in the calcination step. Reducing gases are for example CO-containing atmospheres or a forming gas, preferably argon-hydrogen mixtures or nitrogen-argon mixtures (97/3 and 95/5). For reasons of occupational safety, health protection and environmental protection, such reducing gases are unfavourable. In order to ensure occupational safety on account of the use of these gases for those involved, it is necessary to take precautions, for example by expenditure on apparatus, which in term entails financial cost.

Preferably, the process can be conducted under air atmosphere.

Preferably, in the process according to the invention the amount of flux is not more than 50.0% by weight, preferably not more than 10.0% by weight, particularly preferably not more than 4.0% by weight, based on the total amount of the reactants.

It has been shown that the particle size distribution of the fluxed phosphor according to the invention resembles a Gaussian distribution, which points to the homogeneity of the particle size, and so the incorporation thereof in a coating matrix can advantageously be conducted significantly more easily. It is assumed that the coating properties, such as the appearance of the coating surface, for example the gloss, feel and touch, were improved as a result of this.

The intensity of the emission of the up-conversion phosphors could also be achieved through a simple technical implementation.

Preferred silicon dioxides used may be the products having the trade names Aerosil® 300, 200, OX50, 200 V and 300 V from Evonik.

Preferably, the halogen-containing flux used is at least one substance from the group of the ammonium halides, alkali metal halides, alkaline earth metal halides and lanthanoid halides. It has surprisingly been found with halides from these groups that up-conversion phosphors prepared using them have a higher emission intensity than with other fluxes.

The halides are preferably fluorides or chlorides.

The alkali metals are preferably potassium, sodium or lithium.

The lanthanoid is preferably praseodymium.

The alkaline earth metals are preferably calcium or strontium.

The phosphor is preferably doped with praseodymium in the process according to the invention.

The phosphor is preferably doped with praseodymium and co-doped with gadolinium in the process according to the invention.

The invention further provides an up-conversion phosphor of the general formula (I)

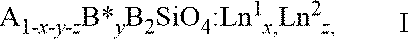

with
x = 0.0001 - 0.0500;
z = 0.0000 or z = 0.0001 to 0.3000 with the proviso that: y = x + z;
A being selected from the group consisting of Mg, Ca, Sr and Ba;
B being selected from the group consisting of Li, Na, K, Rb and Cs;
B* being selected from the group consisting of Li, Na and K, where B is the same as B* or B is not the same as B*, and preferably B and B* are not the same;
Ln¹ being selected from the group consisting of praseodymium (Pr), erbium (Er) and neodymium (Nd);
Ln² being selected from gadolinium (Gd), obtainable by the process according to the invention, wherein it has a specific surface area determined by gas absorption according to Brunauer, Emmett and Teller (BET) of 1 to 500 m²/g, preferably 5 - 250 m²/g, particularly preferably 10 - 100 m²/g, measured to DIN 66131:1993-07.

The phosphor is preferably a crystalline silicate or made from crystalline silicates, doped with lanthanoid ions, comprising at least one alkali metal ion and at least one alkaline earth metal ion.

The phosphor is preferably doped with praseodymium and co-doped with gadolinium.

It is preferable that the phosphor is partially or fully crystalline. The phosphor is thus preferably at least not entirely amorphous. It is therefore preferable that the phosphor is not an amorphously solidified melt (glass). The phosphor preferably has a crystalline fraction of > 50%, preferably of > 70%, particularly preferably of > 85%, calculated according to the calculation formula (DOC = Degree of Crystallinity)

$$D O C = \frac{C r y s t a l l i n e a r e a}{C r y s t a l l i n e a r e a + A m o r p h o u s a r e a}$$

with the aid of an X-ray powder diffractogram. Reference is made to the description of the method.

The phosphor is preferably selected from compounds of the general formula (Ia)

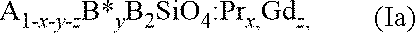

with A being selected from the group consisting of Mg, Ca, Sr, Ba;
B being selected from the group consisting of Li, Na, K, Rb and Cs;
B* being selected from the group consisting of Li, Na and K, where B is the same as B* or B is not the same as B*, and preferably B and B* are not the same;
x = 0.0001 - 0.0500;
z = 0.0000 or z = 0.0001 to 0.3000 with the proviso that: y = x + z.

B* serves here to balance the charge of the praseodymium or gadolinium substitution.

A here may represent a single element from the group consisting of Mg, Ca, Sr and Ba, or else a combination of two or more elements from this group, i.e., for example A = (Mgₐ₁ Caₐ₂ Srₐ₃ Baₐ₄) with 0 ≤ a1 ≤ 1, 0 ≤ a2 ≤ 1, 0 ≤ a3 ≤1, 0 ≤ a4 ≤ 1, and with the proviso that: a1 + a2 + a3 + a4 = 1. A may thus represent (Ca_{0.9}Sr_{0.1}), for example.

The phosphor is preferably selected from compounds of the general formula (II)

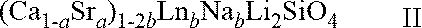

where:
Ln is selected from the group consisting of praseodymium, gadolinium, erbium, neodymium, preferably praseodymium;
a = 0.0000 to 1.0000, preferably 0.0000 to 0.1000, especially 0.0000;
b = 0.0001 to 0.5000, preferably 0.0001 to 0.1000, especially 0.0050 to 0.0500.

Ln here may represent a single element from the group consisting of praseodymium, gadolinium, erbium and neodymium, or else represent a combination of two elements from this group, i.e., for example, Ln = (Ln¹ₓ Ln²_{y}) where Ln¹ and Ln² are selected from the group consisting of praseodymium, gadolinium, erbium and neodymium, and where x and y are as defined for formulae (I) and (Ia).

Ln¹ serves for doping. Preference is given to using praseodymium for the doping. Ln² serves for optional co-doping. Preference is given to using gadolinium for the optional co-doping. The phosphor has preferably not been co-doped; in other words, Ln preferably represents a single element from the group consisting of praseodymium, gadolinium, erbium and neodymium.

It is even more preferable for the phosphor to be selected from compounds of the general formula (IIa)

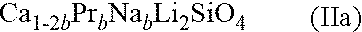

with b = 0.0001 to 0.5000, preferably 0.0001 to 0.1000, especially 0.0050 to 0.0500.

It is very particularly preferable for the phosphor to be Ca_{0.98}Pr_{0.01}Na_{0.01}Li₂SiO₄ or Ca_{0.94}Pr_{0.03}Na_{0.03}Li₂SiO₄ or Ca_{0.90}Pr_{0.05}Na_{0.05}Li₂SiO₄.

Preferably, the up-conversion phosphor according to the invention includes a halogen, corresponding to the halide of the flux.

The phosphor is preferably one which converts electromagnetic radiation having lower energy and longer wavelength in the range from 2000 nm to 400 nm, especially in the range from 800 nm to 400 nm, to electromagnetic radiation having higher energy and shorter wavelength in the range from 400 nm to 100 nm, preferably in the range from 300 nm to 200 nm. It is further preferable for the intensity of the emission maximum of the electromagnetic radiation having higher energy and shorter wavelength to be an intensity of at least 1 • 10³ counts/(mm²*s), preferably higher than 1 • 10⁴ counts/(mm²*s), particularly preferably higher than 1 • 10⁵ counts/(mm²*s). For determination of these indices, emission is preferably induced by means of a laser, especially a laser having a power of 75 mW at 445 nm and/or a power of 150 mW at 488 nm.

The phosphor according to formula (II) preferably has XRPD signals in the range from 23° 2θ to 27° 2θ and from 34° 2θ to 39.5° 2θ, the signals being determined by means of the Bragg-Brentano geometry and Cu-K_{α} radiation. Details of the method of measurement can be found in the as-yet unpublished European patent applications EP 19202910.6 and PCT/EP2020/077798.

PCT/EP2020/077798 is dedicated to the preparation of phosphors, especially of phosphors of formula (I), formula (la) and formula (II), without the addition of fluxes.

Further detailed embodiments of the process can be gathered from EP 19202910.6 and PCT/EP2020/077798, at least 3.5% by weight of flux, based on the total amount of the reactants, being used for the process according to the invention.

Completely surprisingly, it was possible to modify the known process in an elegant manner, additionally leading to optimized up-conversion phosphors with exceptional and unexpected properties with respect to the particle size distribution, increase in the emission intensity and specific surface area.

It is assumed that the addition of more than 3.5% by weight of flux leads to a more homogeneous crystallization/melting process. In this process, the praseodymium ions could be distributed more homogeneously in the lattice and enable a uniform doping. The more homogeneous melting process could furthermore result in sintering of the particle surface and in this way in a lower specific surface area of the up-conversion phosphors. Experience has shown that particles with a lower specific surface area can be incorporated into the coating matrix with lower input of energy.

It has been found that, surprisingly, the phosphors according to the invention, prepared in accordance with the teaching of EP 19202910.6 and PCT/EP2020/077798, have the required up-conversion properties responsible for the antimicrobial action. In other words, these phosphors can convert electromagnetic radiation having wavelengths above UV radiation, especially visible light or infrared light, to electromagnetic radiation having shorter wavelength, specifically in the region in which, for example, the DNA or RNA of the microorganisms can be destroyed or mutated. Accordingly, these phosphors are of very good suitability for the composition according to the invention.

It should be mentioned here that it is possible to use a subsequent milling of the phosphor in accordance with the teaching of EP 19202910.6 and PCT/EP2020/077798 to firstly achieve homogeneity of the particle size and secondly to achieve the desired particle size. However, in this case the energy input would be higher and the milling process would last longer due to the inhomogeneity and particle size distribution thereof after the preparation.

The invention also provides for the use of the phosphors prepared by the process according to the invention for the production of coatings having an antimicrobial property comprising
at least one film-forming polymer,
optionally at least one additive,
optionally at least one curing agent.

The selection of film-forming polymers plays an important role here. In principle, all film-forming polymers known from the prior art are useful.

The film-forming polymer preferably has functional groups, preferably acidic hydrogens, that are reactive with an isocyanate-containing curing agent, and is optionally catalysed by a catalyst.

Advantageously, the film-forming polymer is selected from the group of the hydroxy-functional acrylate polymers, hydroxy-functional polyester polymers, and/or hydroxy-functional polyether polymers, hydroxy-functional cellulose derivatives, amino-functional aspartic polymers or polyester polymers, which reacts with an isocyanate-containing curing agent.

The film-forming polymer preferably has low resonance.

The person skilled in the art is aware of the physical interactions at the surface. Depending on the material and its material surface, a plurality of effects occur at the surface on incidence of light. The incident light is partly absorbed, partly reflected and, depending on the material surface, also scattered. Light can also first be absorbed and then emitted again. In the case of opaque, semitransparent or transparent materials, the light can also penetrate through the body (transmission). In some cases, the light is even polarized or diffracted at the surface. Some objects can even emit light (illuminated displays, LED segments, display screens), or fluoresce or phosphoresce in light of a different colour (afterglow).

What is meant by “low resonance” in the context of the present invention is that the film-forming polymer has low absorption, reflection, remission and scatter in the UV region. By contrast, transmittance should preferably be pronounced.

This is because it has been found that, surprisingly, the film-forming polymers according to the invention that have low resonance have improved antimicrobial action, because more electromagnetic radiation having lower energy and higher wavelength in the range from 2000 nm to 400 nm, especially in the range from 800 nm to 400 nm, is transmitted and, as a result, can be converted to more electromagnetic radiation having higher energy and shorter wavelength in the range from 400 nm to 100 nm, preferably in the range from 300 nm to 200 nm.

It has been found that the higher the transmittance, the higher the emission as well, which is crucial for antimicrobial action.

Preferably, the transmittance of the film-forming polymer is at least 75%, preferably at least 80% and particularly preferably at least 85%, measured at a wavelength of 260 nm.

Preferably, the transmittance of the film-forming polymer is at least 75%, preferably at least 80% and particularly preferably at least 85%, measured at a wavelength of 500 nm.

By way of illustration, it should be noted here that transmittance may be defined at a different wavelength; see the FIGURE. For the present invention, the wavelengths of 260 nm by way of example for the wavelength emitted and 500 nm by way of example for the excitation wavelength were chosen, which are responsible on the one hand for the up-conversion and on the other hand to a significant degree for the antimicrobial action.

In the case of 100% transmittance, for example, measured at a wavelength of 260 nm, the same amount of radiation is converted and emitted; in other words, there are no losses through absorption, scatter or the like. In the case of 80% transmittance, measured at a wavelength of 260 nm, 20% is not transmitted, probably owing to absorption, reflection, remission and/or scatter. Accordingly, only 80% of the radiation of wavelength 260 nm can be emitted.

This significant finding is important in the selection of the film-forming polymers. Polymers having 0% transmittance, for example, are unsuitable for the curable composition according to the invention. They do not transmit any electromagnetic radiation having lower energy and higher wavelength and, accordingly, phosphors present in the composition cannot convert this electromagnetic radiation to electromagnetic radiation having higher energy and shorter wavelength and emit it, which is required for the antimicrobial action.

Preferably, the composition according to the invention has a transmittance of at least 75%, preferably at least 80% and particularly preferably at least 85%, measured at 260 nm.

Preferably, the composition according to the invention has a transmittance of at least 75%, preferably at least 80% and particularly preferably at least 85%, measured at 500 nm.

The transmittance curves are preferably measured with a “Specord 200 Plus” twin-beam UV/VIS spectrometer from Analytik Jena. A holmium oxide filter is used for internal wavelength calibration. Monochromatic light from a deuterium lamp (UV range) or a tungsten-halogen lamp (visible range) is passed through the samples. The spectral range is 1.4 nm. The monochromatic light is divided into a measurement channel and a reference channel and enables direct measuring against a reference sample. The radiation transmitted through the sample is detected by a photodiode and processed to form electrical signals.

It is conceivable to use a composition having a low transmittance of less than 70%; they possibly also still have antimicrobial action, but the efficiency is very moderate.

The phosphors preferably have an average particle size of d50 of 0.1 - 50 µm, preferably d50 = 0.1 - 25 µm, particularly preferably d50 = 0.1 µm - 5 µm, measured to ISO 13320:2020 and USP 429, for example with an LA-950 Laser Particle Size Analyzer from Horiba.

In order to efficiently incorporate and/or stabilize the phosphors in the composition according to the invention, it is preferably possible to add various additives.

The additives are preferably selected from the group of the dispersants, rheology aids, levelling agents, wetting agents, defoamers and UV stabilizers.

It has been found that, surprisingly, any addition of additives to the composition according to the invention reduces transmittance.

Accordingly, the composition according to the invention, in a further embodiment in which additives are used, preferably has a transmittance of at least 70%, preferably at least 75% and particularly preferably at least 80%, measured at 260 nm.

Accordingly, the composition according to the invention, in a further embodiment in which additives are used, preferably has a transmittance of at least 70%, preferably at least 75% and particularly preferably at least 80%, measured at 500 nm.

Preferably, the composition according to the invention includes a curing agent selected from the group of the aliphatic or cycloaliphatic isocyanates.

Examples of isocyanate-containing curing agents are monomeric isocyanates, polymeric isocyanates and isocyanate prepolymers. Polyisocyanates are preferred over monomeric isocyanates on account of their lower toxicity. Examples of polyisocyanates are isocyanurates, uretdiones and biurets based on diphenylmethane diisocyanate (MDI), toluene diisocyanate (TDI), hexamethylene diisocyanates (HDI) and isophorone diisocyanate (IPDI). Examples of commercially available products are those under the trade name DESMODUR® from Covestro or VESTANAT from Evonik Industries. Known products are DESMODUR® N3400, DESMODUR® N3300, DESMODUR® N3600 DESMODUR® N75, DESMODUR® XP2580, DESMODUR® Z4470, DESMODUR® XP2565 and DESMODUR® VL from Covestro. Further examples are VESTANAT® HAT 2500 LV, VESTANAT® HB 2640 LV or VESTANAT® T 1890E from Evonik Industries. Examples of isocyanate prepolymers are DESMODUR® E XP 2863, DESMODUR® XP 2599 or DESMODUR® XP 2406 from Covestro. Further isocyanate prepolymers known to the person skilled in the art may be used.

It is conceivable to use catalysts for the curing. The catalysts that follow, selected from organic Sn(IV), Sn(II), Zn, Bi compounds or tertiary amines, may be used.

Preference is given to using catalysts selected from the group of organotin catalysts, titanates or zirconates, organometallic compounds of aluminium, iron, calcium, magnesium, zinc or bismuth, Lewis acids or organic acids/bases, linear or cyclic amidines, guanidines or amines or a mixture thereof.

Curing catalysts used are preferably organic tin compounds, for example, dibutyltin dilaurate, dibutyltin diacetylacetonate, dibutyltin diacetate, dibutyltin dioctoate, or dioctyltin dilaurate, dioctyltin diacetylacetonate, dioctyltin diketanoate, dioctylstannoxane, dioctyltin dicarboxylate, dioctyltin oxide, preferably dioctyltin diacetylacetonate, dioctyltin dilaurate, dioctyltin diketanoate, dioctylstannoxane, dioctyltin dicarboxylate, dioctyltin oxide, particularly preferably dioctyltin diacetylacetonate and dioctyltin dilaurate. In addition, it is also possible to use zinc salts, such as zinc octoate, zinc acetylacetonate and zinc 2-ethylcaproate, or tetraalkylammonium compounds, such as N,N,N-trimethyl-N-2-hydroxypropylammonium hydroxide, N,N,N-trimethyl-N-2-hydroxypropylammonium 2-ethylhexanoate or choline 2-ethylhexanoate. Preference is given to the use of zinc octoate (zinc 2-ethylhexanoate) and of the tetraalkylammonium compounds, particular preference to that of zinc octoate. Further preferred are bismuth catalysts, e.g. TIB Kat (TIB Mannheim) or Borchi® catalysts, titanates, e.g. titanium(IV) isopropoxide, iron(III) compounds, e.g. iron(III) acetylacetonate, aluminium compounds, such as aluminium triisopropoxide, aluminium tri-sec-butoxide and other alkoxides and also aluminium acetylacetonate, calcium compounds, such as calcium disodium ethylenediaminetetraacetate or calcium diacetylacetonate, or else amines, examples being triethylamine, tributylamine, 1,4-diazabicyclo[2.2.2]octane, 1,8-diazabicyclo[5.4.0]undec-7-ene, 1,5 diazabicyclo[4.3.0]non-5-ene, N,N-bis(N,N-dimethyl-2-aminoethyl)methylamine, N,N-dimethylcyclohexylamine, N,N-dimethylphenylamine, N-ethylmorpholine, etc. Also preferred as catalysts are organic or inorganic Brønsted acids such as acetic acid, trifluoroacetic acid, methanesulfonic acid, p-toluenesulfonic acid or benzoyl chloride, hydrochloric acid, phosphoric acid and the monoesters and/or diesters thereof, for example butyl phosphate, (iso)propyl phosphate, dibutyl phosphate, etc. Also preferred are guanidine-bearing organic and organosilicon compounds. It is of course also possible to use combinations of two or more catalysts. In addition, it is also possible to use photolatent bases as catalysts, as described in WO 2005/100482.

The curing catalyst is preferably used in amounts of 0.01% to 5.0% by weight, preferably 0.05% to 4.0% by weight and particularly preferably 0.1% to 3% by weight, based on the total weight of the curable composition.

In the case of film-forming polymers that cure through physical drying, the addition of reactive curing agents is not required.

The composition according to the invention may preferably be used in 1 K (one-component) coating systems or 2 K (two-component) coating systems, in melamine baking systems, or room- or high-temperature systems.

Preferably, coatings produced from the composition according to the invention have antimicrobial action against bacteria, yeasts, moulds, algae, parasites and viruses.

The coatings produced according to the invention preferably have antimicrobial action against
pathogens of nosocomial infections, preferably against Enterococcus faecium, Staphylococcus aureus, Klebsiella pneumoniae, Acinetobacter baumannii, Pseudomonas aeruginosa, Escherichia coli, Enterobacter, Corynebacterium diphtheriae, Candida albicans, rotavirus, bacteriophages;
facultatively pathogenic environmental organisms, preferably against Cryptosporidium parvum, Giardia lamblia, amoebas (Acanthamoeba spp., Naegleria spp.), E. coli, coliform bacteria, faecal streptococci, Salmonella spp., Shigella spp., Legionella spec., Pseudomonas aeruginosa, Mycobacterium spp., enteral viruses (e.g. polio and hepatitis A virus);
pathogens in food and drink, preferably against Bacillus cereus, Campylobacter spp., Clostridium botulinum, Clostridium perfringens, Cronobacter spp., E. coli, Listeria monocytogenes, Salmonella spp., Staphylococcus aureus, Vibrio spp., Yersinia enterocolitica, bacteriophages.

It has been found that the incorporation of the up-conversion phosphors according to the invention was markedly improved.

Up-conversion phosphors and phosphors are used as synonyms.

The invention further provides for the use of the phosphors in compositions for the production of dispersions, millbases, adhesives, trowelling compounds, renders, paints, coatings or printing inks, inkjets, grinding resins or pigment concentrates.

Preference is given to the use of the composition according to the invention for the production of coatings having an antimicrobial property.

What is meant here by a coating having antimicrobial action or an antimicrobial property is that the coating has an antimicrobial surface that limits or prevents the growth and propagation of microorganisms.

It has also been found that, astonishingly, the coatings according to the invention have chemical and mechanical stability. Chemical and mechanical stability is particularly important since antimicrobial coatings are frequently used in areas that require regular disinfection and further hygiene measures.

The invention also includes a process for forming an antimicrobial coating on a substrate, comprising the application of a curable film-forming composition to the substrate, comprising:
a. at least one film-forming polymer containing functional groups which are reactive with an isocyanate-containing curing agent, optionally catalysed by a catalyst,
b. at least one phosphor of the formula (II) and
c. a curing agent containing isocyanate-functional groups.

Preferably, the substrate is metal, mineral substrates (for instance concrete, natural rock or glass), cellulosic substrates, wood and hybrids thereof, dimensionally stable plastics and/or thermosets.

The term “dimensionally stable plastics” is understood to mean, albeit non-exhaustively, the following polymers: acrylonitrile-butadiene-styrene (ABS), polyamides (PA), polylactate (PLA), polymethyl methacrylate (PMMA), polycarbonate (PC), polyethylene terephthalate (PET), polystyrene (PS), polyether ether ketone (PEEK), polyvinyl chloride (PVC), polypropylene (PP), polyethylene (PE).

Preferably, a primer composition may be applied to the substrate prior to the application of the curable film-forming composition.

Preferably, the curable composition according to the invention is used for the coating of substrates in hygiene facilities and hospitals and in the food and drink industry.

This includes all settings in the public sphere, for example schools, old people’s homes, industrial kitchens or nurseries.

A further invention is an article that has been coated at least partly, preferably fully, with the curable composition according to the invention.

It should be noted here that the terms “antimicrobial effect”, “antimicrobial efficacy”, “antimicrobial action” and “antimicrobial property” are used as synonyms.

It should be noted here that the article according to the invention may preferably have antimicrobial action even without release of an antimicrobial active ingredient if the coating comprises specific phosphors as described. In this way, the route via which the microorganisms are then killed is physical. Therefore, such materials are not covered by the biocide regulation (Regulation (EU) No 528/2012 of the European Parliament and of the Council of 22 May 2012 in the current text of 2019).

Adduced hereinafter are examples that serve solely to elucidate this invention to the person skilled in the art and do not constitute any restriction at all of the subject-matter as described.

### Methods

Particle size distribution to ISO 13320:2020 and USP 429, with a Horiba LA-950 Laser Particle Size Analyzer

Qualitative elemental analysis by means of EDX with a Tabeltop 4000Plus from Hitachi, 15 kV BSE detector, 1000x magnification

Powder XRD: The X-ray powder diffractograms of the samples were recorded using a Bruker D2 Phaser powder diffractometer operating in Bragg-Brentano geometry, using Cu-K_{α} radiation and a line scan CCD detector. The integration time was 20 s and the step width was 0.017° 2θ.

The emission spectra were recorded with the aid of an Edinburgh Instruments FLS920 spectrometer equipped with a 488 nm continuous-wave OBIS laser from Coherent and a Peltier-cooled (-20° C.) single-photon counting photomultiplier from Hamamatsu (R2658P). Edge filters were used to suppress second- and higher-order reflections caused by the monochromators.

BET surface area measurements to ISO 9277, DIN 66131 using a Nova 2000e instrument from Quantachrome.

The degree of crystallinity (DOC) gives information on the ratio of the crystalline area to the amorphous area of all components in a powder diffractogram, as described above in the Powder XRD section. The degree of crystallinity is calculated from the total area under the crystalline and amorphous fractions:

$$D O C = \frac{C r y s t a l l i n e a r e a}{C r y s t a l l i n e a r e a + A m o r p h o u s a r e a}$$

### Phosphors

### Example 1 Phosphor According to the Invention (Ca_{0.98}Pr_{0.01}Na_{0.01})Li₂SiO₄ With 4% by Weight of CaF₂ as Flux

4.12 g of CaCO₃, 3.11 g of Li₂CO₃, 2.52 g of SiO₂, 0.02 g of Na₂CO₃, 0.07 g of Pr₆O₁₁, and 0.4 g of CaF₂ were mixed with one another. This mixture was calcined at 850° C. for 6 h in air, which results in the desired product. The phosphor was withdrawn for further measurements.

BET: 3 m²/g
Particle size distribution:

D₁₀: 3 µm
D₅₀: 9 µm
D₉₀: 32 µm

Degree of crystallinity: 89%

### Example 2 Phosphor According to the Invention (Ca_{0.98}Pr_{0.01}Na_{0.01})Li₂SiO₄ With 6% by Weight of CaF₂ as Flux

4.12 g of CaCO₃, 3.11 g of Li₂CO₃, 2.52 g of SiO₂, 0.02 g of Na₂CO₃, 0.07 g of Pr₆O₁₁, and 0.62 g of CaF₂ were mixed with one another. This mixture was calcined at 850° C. for 6 h in air, which results in the desired product. The phosphor was withdrawn for further measurements.

BET: 2 m²/g
Particle size distribution:

D₁₀: 3 µm
D₅₀: 10 µm
D₉₀: 60 µm

Degree of crystallinity: 90%

### Comparative Example: Phosphor (Ca_{0.98}Pr_{0.01}Na_{0.01})Li₂SiO₄ With 1.5% by Weight of CaF₂ as Flux

4.12 g of CaCO₃, 3.11 g of Li₂CO₃, 2.52 g of SiO₂, 0.02 g of Na₂CO₃, 0.07 g of Pr₆O₁₁, and 0.15 g of CaF₂ were mixed with one another. This mixture was calcined at 850° C. for 6 h in air, which results in the desired product. The phosphor was withdrawn for further measurements.

BET: 49 m²/g
Particle size distribution:

D10: 3 µm
D50: 12 µm
D90: 56 µm

Degree of crystallinity: 93%

The particle size distribution of the phosphors according to the invention (Examples 1 and 2) and the comparative example do not exhibit any significant change. The addition of 4% by weight or 6% by weight of CaF₂ results in a significant reduction in the specific surface area (BET) of the phosphors according to the invention (Examples 1 and 2) compared to the phosphor comprising 1.5% by weight. A reduction in the BET surface area with simultaneously stable particle size distribution is indicative of a reduction in the porosity. The degree of crystallinity of the phosphors does not change significantly as a result of the addition of increased CaF₂ admixtures.

All phosphors exhibited an up-conversion property in the emission spectrum in the UV-C region and an antimicrobial effect. The incorporation of the phosphors according to the invention into the coating matrix was much easier.

The FIGURE shows an emission spectrum for Examples 1 and 2 and for the comparative example. The phosphors exhibited the desired wavelength range.

1. A process for the preparation of an up-conversion phosphor of the general formula (I) 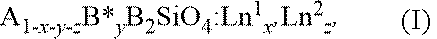
wherein
x = 0.0001 - 0.0500;
z = 0.0000 or z = 0.0001 to 0.3000 with the proviso that y = x + z;
A is selected from the group consisting of Mg, Ca, Sr, and Ba;
B is selected from the group consisting of Li, Na, K, Rb, and Cs;
B* is selected from the group consisting of Li, Na, and K,
Ln¹ is selected from the group consisting of praseodymium (Pr), erbium (Er),
and neodymium (Nd); and
Ln² is gadolinium (Gd),
the process comprising:
providing the following components:
i) at least one of
a lanthanoid salt selected from the group consisting of a lanthanoid nitrate, a lanthanoid carbonate, a lanthanoid carboxylate, and a lanthanoid sulfate, and/or
a lanthanoid oxide,
wherein a lanthanoid ion in the lanthanoid oxide or lanthanoid salt is selected from praseodymium, gadolinium, erbium, and neodymium; and at least two of the lanthanoid ions for co-doping,
ii) a silicate or a silicon dioxide,
iii) at least one alkaline earth metal salt, and
at least one alkali metal salt selected from a lithium salt or a lithium compound, and optionally selected from a sodium salt and potassium salt, and
iv) at least one flux selected from the group consisting of ammonium halide, alkali metal halide, alkaline earth metal halide, and lanthanoid halide,
a) mixing components i), ii), iii) and iv) by grinding to obtain a mixture, or
b) mixing components i), ii), iii) and iv) in an organic polar or nonpolar solvent that is not a protic solvent by grinding to obtain a mixture;
c) precalcining the mixture from b) at 600 to 1000° C., to remove an organic component, for at least 1 h, under air atmosphere to obtain a precalcined mixture, and optionally cooling to room temperature; and
d) calcining the mixture from a) or the precalcined mixture from c) at a temperature from 600 to < 1000° C., for at least 3 h, and
e) obtaining a silicate-based up-conversion phosphor of the general formula (I), wherein at least 3.5% by weight of the at least one flux is used, based on a total amount of reactants.

2. The process according to claim 1, wherein an amount of the at least one flux is not more than 50.0% by weight, based on the total amount of the reactants.

3. The process according to claim 1, wherein d) is conducted under air atmosphere.

4. The process according to claim 1, wherein the lanthanoid is praseodymium.

5. The process according to claim 1, wherein alkali metals are sodium and/or lithium.

6. The process according to claim 1, wherein an alkaline earth metal is calcium.

7. The process according to claim 1, wherein the silicate-based up-conversion phosphor of the general formula (I) is doped with praseodymium.

8. A silicate-based up-conversion phosphor of the general formula (I) 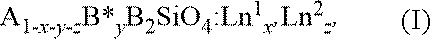
wherein
x = 0.0001 - 0.0500;
z = 0.0000 or z = 0.0001 to 0.3000 with the proviso that y = x + z;
A is selected from the group consisting of Mg, Ca, Sr, and Ba;
B is selected from the group consisting of Li, Na, K, Rb, and Cs;
B* is selected from the group consisting of Li, Na, and K;
Ln¹ is selected from the group consisting of praseodymium (Pr), erbium (Er), and neodymium (Nd);
Ln² is gadolinium (Gd),
obtainable by the process according to claim 1, wherein the phosphor has a specific surface area determined by gas absorption according to Brunauer, Emmett and Teller (BET) of 1 to 500 m²/g, measured to ISO 9277, DIN 66131.

9. The phosphor according to claim 8, wherein the phosphor has been doped with praseodymium and co-doped with gadolinium.

10. The phosphor according to claim 8, wherein the phosphor is a solidified melt composed of crystalline silicates or of crystalline silicates doped with lanthanoid ions, comprising at least one alkali metal ion and at least one alkaline earth metal ion.

11. The phosphor according to claim 8, wherein the phosphor is at least partially crystalline.

12. The phosphor according to claim 8, wherein the phosphor is a compound of the general formula (Ia) 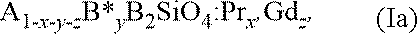
wherein
A is selected from the group consisting of Mg, Ca, Sr, and Ba;
B is selected from the group consisting of Li, Na, K, Rb, and Cs;
B* is selected from the group consisting of Li, Na, and K;
x = 0.0001 - 0.0500; and
z = 0.0000 or z = 0.0001 to 0.3000 with the proviso that: y = x + z.

13. The phosphor according to claim 8, wherein the phosphor is a compound of the general formula (II) 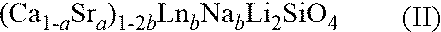
wherein
Ln is selected from the group consisting of praseodymium, gadolinium, erbium, and neodymium;
a = 0.0000 to 1.0000; and
b = 0.0001 to 0.5000.

14. The phosphor according to claim 8, wherein the phosphor is a compound of the general formula (IIa) 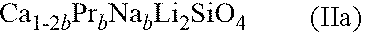
wherein b = 0.0001 to 1.

15. The phosphor according to claim 8, wherein the phosphor is 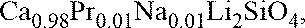 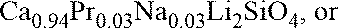 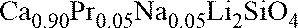
.

16. The phosphor according to claim 13, wherein the compound according to formula (II) has XRPD signals in the range from 23° 2θ to 27° 2θ and from 34° 2θ to 39.5° 2θ.

17. A method for production of a coating having an antimicrobial property, the method comprising:
applying a composition to a substrate,
wherein the composition comprises
the phosphor according to claim 8,
at least one film-forming polymer,
optionally, at least one additive, and
optionally, at least one curing agent.

18. The process according to claim 1, wherein the lanthanoid oxide is Pr₆O₁₁ and/or Gd₂O₃.

19. The process according to claim 1, wherein the at least one alkaline earth metal salt is calcium carbonate, and the at least one alkali metal salt is lithium carbonate and sodium carbonate.

20. The process according to claim 1, wherein the at least one flux is selected from the group consisting of ammonium chloride, sodium chloride, sodium fluoride, sodium bromide, lithium fluoride, lithium chloride, calcium chloride, calcium fluoride, praseodymium fluoride, and praseodymium chloride.
